(19) United States
(12) Patent Application Publication
(10) Pub. No.: US 2007/0099223 A1
(43) Pub. Date: May 3, 2007

(54) Single nucleotide polymorphism associated with stroke susceptibility

(76) Inventors: Steven J. Kittner, Ellicott City, MD (US); Qing Song, Decatur, GA (US); Braxton D. Mitchell, Towson, MD (US); Jeffrey R. O'Connell, Ellicott City, MD (US); John W. Cole, Baltimore, MD (US); O. Colin Stine, Baltimore, MD (US); Gary H. Gibbons, Atlanta, GA (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 11/589,487

(22) Filed: Oct. 30, 2006

Related U.S. Application Data
(60) Provisional application No. 60/731,402, filed on Oct. 28, 2005.
(60) Provisional application No. 60/733,334, filed on Nov. 3, 2005.

(51) Int. Cl. C12Q 1/68

(52) U.S. Cl. 435/6

(57) ABSTRACT

The present invention identifies various risk alleles, particularly a risk allele of SNP rs918592, within the PDE4D gene as novel stroke associated risk markers. These markers may be used for identifying a subject's susceptibility to stroke and diagnosing a subject's susceptibility to a particular type of stroke based on the presence of the risk allele. The markers are found in the human population, particularly the human female population, across different ethnicities. The marker further reveals an environmental impact on a subject's susceptibility to stroke, showing a dose dependent relationship.

### CROSS REFERENCE TO RELATED APPLICATION

The present application claims priority under 35 U.S.C. §119 to the U.S. Provisional Application Ser. No. 60/731,402, filed on Oct. 28, 2005, and U.S. Provisional Application Ser. No. 60/733,334, filed on Nov. 3, 2005. U.S. Provisional Applications 60/731,402 and 60/733,334 are herein incorporated by reference in their entireties.

### ACKNOWLEDGEMENT OF GOVERNMENT LICENSE RIGHTS

The U.S. Government has a paid-up license in this invention and the right in limited circumstances to require the patent owner to license others on reasonable terms as provided for by the terms of Grant No. P60 12583 awarded by the National Institute on Aging Pepper Center. This work was also supported, in whole or in part, by funding from the Department of Veterans Affairs, Veterans Administration Medical Center, Baltimore. Additionally, support for the research disclosed herein was provided by the Maryland Cigarette Restitution Fund Program, University of Maryland School of Medicine, Baltimore, Morehouse School of Medicine, Johns Hopkins Bloomberg School of Public Health, and Centers for Disease Control.

### FIELD OF THE INVENTION

The present invention generally relates to the field of vascular disease and particularly to the association between allelic polymorphisms within genes and stroke in certain populations. More particularly, associations between polymorphisms within the phosphodiesterase 4D gene (PDE4D) and ischemic stroke.

### BACKGROUND OF THE INVENTION

Strokes are a leading cause of death and disability, not only in the USA but around the world. Thus, an area of significant interest is the prevention, diagnosis and/or treatment of strokes. In general, stroke events may be classified as either hemorrhagic or ischemic in nature, with ischemic events (and its subtypes, such as atherosclerotic and cardioembolic) being the most common type of stroke events. Cerebral infarction (ischemic stroke) and other types of stroke have been tied with the concept of genetic risk factors, wherein the presence of certain alleles (“risk alleles”) of genes within chromosomes may indicate an individual's predisposition for experiencing a stroke event. These risk alleles may often be expressed as or created through single nucleotide polymorphisms (SNPs) within one allele of the chromosome gene pair. This allelic variance (genetic risk factor) between the gene pair on the chromosome provides a marker that may allow for identification of the presence of the risk factor. Therefore, the identification and detection of these various genetic risk factors is believed to be critical to the diagnosis and treatment and/or prevention of stroke events.

The phosphodiesterase 4D gene (PDE4D) has been implicated in the etiology of stroke, particularly the atherosclerotic and cardioembolic subtypes based on linkage and association analyses carried out by deCODE Genetics in the Icelandic population. Gretarsdottir S, Thorleifsson G, Reynisdottir S T, et al. The gene encoding phosphodiesterase 4D confers risk of ischemic stroke. Nat Genet 35: 131-138, 2003. The human PDE4D gene spans a 1.6 Mbp region on chromosome 5q12 and contains 24 exons (OMIM 600129). Through differential promoter and alternative splicing in gene expression, the gene can express 9 different functional protein isoforms (FIG. 1). Associated SNPs have been located among the first exons at the 5′end of the gene. The association with stroke may be mediated through cAMP, a key signal transduction molecule in multiple tissues and cell types, including vascular cells. Dominiczak A F, McBride M W. Genetics of common polygenic stroke. Nat Genet 35: 116-117, 2003. PDE4D selectively degrades cAMP, which exerts protean effects on the vasculature and nervous system.

A Swedish (Nilsson-Ardnor S, Wiklund P G, Lindgren P, Nilsson A K, Janunger T, Escher S A, Hallbeck B, Stegmayr B, Asplund K, Holmberg D. Linkage of ischemic stroke to the PDE4D region on 5q in a Swedish population. Stroke. 2005 August; 36(8):1666-71), but not an American (Meschia J F, Brott T G, Brown R D Jr, Crook R, Worrall B B, Kissela B, Brown W M, Rich S S, Case L D, Evans E W, Hague S, Singleton A, Hardy J. Phosphodiesterase 4D and 5-lipoxygenase activating protein in ischemic stroke. Ann Neurol. 2005 September; 58(3):351-61), study replicated linkage to the PDE4D region of chromosome 5q12. Several studies have examined the deCODE markers in the region of PDE4D where associations were found in Iceland. Lohmussaar E, Gschwendtner A, Mueller J C, et al. ALOX5AP gene and the PDE4D gene in a central European population of stroke patients. Stroke. 2005 April; 36(4):731-6. Epub Feb. 24, 2005. Some (Nilsson-Ardnor S, Wiklund P G, Lindgren P, Nilsson A K, Janunger T, Escher S A, Hallbeck B, Stegmayr B, Asplund K, Holmberg D. Linkage of ischemic stroke to the PDE4D region on 5q in a Swedish population. Stroke. 2005 August; 36(8):1666-71; Meschia J F, Brott T G, Brown R D Jr, Crook R, Worrall B B, Kissela B, Brown W M, Rich S S, Case L D, Evans E W, Hague S, Singleton A, Hardy J. Phosphodiesterase 4D and 5-lipoxygenase activating protein in ischemic stroke. Ann Neurol. 2005 September; 58(3):351-61; Bevan S, Porteous L, Sitzer M, et al. Phosphodiesterase 4D Gene, Ischemic Stroke, and Asymptomatic Carotid Atherosclerosis. Stroke. 2005 May; 36(5):949-53. Epub Mar. 31, 2005; van Rijn M J, Slooter A J, Schut A F, Isaacs A, Aulchenko Y S, Snijders P J, Kappelle L J, van Swieten J C, Oostra B A, van Duijn C M. Familial aggregation, the PDE4D gene, and ischemic stroke in a genetically isolated population. Neurology. Sep. 14, 2005; [Epub ahead of print]; and Saleheen D, Bukhari S, Haider S R, Nazir A, Khanum S, Shafqat S, Anis M K, Frossard P. Association of Phosphodiesterase 4D Gene With Ischemic Stroke in a Pakistani Population. Stroke. 2005 October; 36(10):2275-7) but not all (Lohmussaar E, Gschwendtner A, Mueller J C, et al. ALOX5AP gene and the PDE4D gene in a central European population of stroke patients. Stroke. 2005 April; 36(4):731-6. Epub Feb. 24, 2005) studies have found associations in this region to all ischemic stroke or some stroke subtypes. All studies to date have examined predominantly older and Caucasian populations and, with one exception (Nilsson-Ardnor S, Wiklund P G, Lindgren P, Nilsson A K, Janunger T, Escher S A, Hallbeck B, Stegmayr B, Asplund K, Holmberg D. Linkage of ischemic stroke to the PDE4D region on 5q in a Swedish population. Stroke. 2005 August; 36(8):1666-71) have examined only deCODE markers.

Replication studies of the Icelandic (Lohmussaar E, Gschwendtner A, Mueller J C, et al. ALOX5AP gene and the PDE4D gene in a central European population of stroke patients. Stroke. 2005 April; 36(4):731-6. Epub Feb. 24, 2005) findings have yielded mixed results. For example, four European studies exclusively examined the strongest genotype and haplotype associations with all ischemic stroke or the combined atherosclerotic/cardioembolic group from the Iceland study and failed to replicate these findings. Nilsson-Ardnor S, Wiklund P G, Lindgren P, Nilsson A K, Janunger T, Escher S A, Hallbeck B, Stegmayr B, Asplund K, Holmberg D. Linkage of ischemic stroke to the PDE4D region on 5q in a Swedish population. Stroke. 2005 August; 36(8):1666-71. Lohmussaar E, Gschwendtner A, Mueller J C, et al. ALOX5AP gene and the PDE4D gene in a central European population of stroke patients. Stroke. 2005 April; 36(4):731-6. Epub Feb. 24, 2005. Bevan S, Porteous L, Sitzer M, et al. Phosphodiesterase 4D Gene, Ischemic Stroke, and Asymptomatic Carotid Atherosclerosis. Stroke. 2005 May; 36(5):949-53. Epub Mar. 31, 2005. A German study examined SNP 41(rs152312), SNP 45(rs12188950), and 1 microsatellite marker (AC008818-1), which had shown the most significant association with stroke in Iceland. Lohmussaar E, Gschwendtner A, Mueller J C, et al. ALOX5AP gene and the PDE4D gene in a central European population of stroke patients. Stroke. 2005 April; 36(4):731-6. Epub Feb. 24, 2005. They further examined a set of haplotype tagged SNPs that distinguished 95% of all chromosomes within linkage disequilibrium blocks “B” and “C”, which contained the haplotypes with the strongest association with stroke in Iceland. No positive associations with ischemic stroke were found.

An English study examined the positively associated Icelandic SNPs and haplotypes in relationship to carotid intima-media thickness and carotid plaque as well as risk of ischemic stroke and ischemic stroke subtypes. Bevan S, Porteous L, Sitzer M, et al. Phosphodiesterase 4D Gene, Ischemic Stroke, and Asymptomatic Carotid Atherosclerosis. Stroke. 2005 May; 36(5):949-53. Epub Mar. 31, 2005. No SNPs or haplotyes were associated with all ischemic stroke or with the combined atherosclerotic/cardioembolic groups. Two SNPs, SNP 19(rs4133470) and SNP 87(rs2910829) were found to be associated with large vessel stroke after adjustment for other risk factors and one of these two, SNP 87(rs2910829), was associated with carotid intima-media thickness at the carotid bulb. Six deCODE SNPs (SNP2(rs152341), SNP13(rs26949), SNP 14(rs26950), SNP 15(rs35382), SNP 20(rs16878206), SNP 26(rs40512)) showed significant association with cardioembolic stroke after multivariate adjustment and haplotypes derived from these SNPs were less strongly associated than the individual SNPs.

A Swedish study (Nilsson-Ardnor S, Wiklund P G, Lindgren P, Nilsson A K, Janunger T, Escher S A, Hallbeck B, Stegmayr B, Asplund K, Holmberg D. Linkage of ischemic stroke to the PDE4D region on 5q in a Swedish population. Stroke. 2005 August; 36(8):1666-71) of predominantly ischemic strokes found no association with the deCODE-associated variants, the A allele of AC008818-1 marker and deCODE SNPs 45(rs12188950) and 41(rs152312) near PDE4D7 region.

A study in a geographically isolated population in the Netherlands (van Rijn M J, Slooter A J, Schut A F, Isaacs A, Aulchenko Y S, Snijders P J, Kappelle L J, van Swieten J C, Oostra B A, van Duijn C M. Familial aggregation, the PDE4D gene, and ischemic stroke in a genetically isolated population. Neurology. Sep. 14, 2005; [Epub ahead of print]) examined deCODE SNPs 39(rs3887175), 45(rs12188950), and 83(rs966221) their derived haplotypes and found no associations with all ischemic stroke either in the total population or within inbred individuals. The subgroup of inbred individuals with small vessel disease did show an association with SNPs 39(rs3887175) and 45(rs12188950).

In contrast, recent American (Meschia J F, Brott T G, Brown R D Jr, Crook R, Worrall B B, Kissela B, Brown W M, Rich S S, Case L D, Evans E W, Hague S, Singleton A, Hardy J. Phosphodiesterase 4D and 5-lipoxygenase activating protein in ischemic stroke. Ann Neurol. 2005 September; 58(3):351-61) and Pakistani (Saleheen D, Bukhari S, Haider S R, Nazir A, Khanum S, Shafqat S, Anis M K, Frossard P. Association of Phosphodiesterase 4D Gene With Ischemic Stroke in a Pakistani Population. Stroke. 2005 October; 36(10):2275-7) studies found significant associations between deCODE SNPs and all ischemic stroke. The American study, including predominately Caucasians, found associations with 2 of 4 deCODE-associated variants, SNP 83(rs966221) and SNP 56(rs702553), with the more strongly associated SNP 83(rs966221) with a stronger association with large artery atherosclerotic stroke. The Pakistani study (Saleheen D, Bukhari S, Haider S R, Nazir A, Khanum S, Shafqat S, Anis M K, Frossard P. Association of Phosphodiesterase 4D Gene With Ischemic Stroke in a Pakistani Population. Stroke. 2005 October; 36(10):2275-7) examined SNP32(rs456009), 83(rs966221), and 87(rs2910829) and found an association with SNP83(rs966221).

Unfortunately, despite the extensive research in this area, the precise functional variants of the PDE4D gene conferring the risk of ischemic stroke have not been identified. The significant discrepancies in the reports to date and the genetic complexity of PDE4D expression have led to uncertainty.

Therefore, it would be desirable to provide for the identification of PDE4D variant(s) that may promote the effective, accurate and/or precise detection of stroke susceptibility in a subject. It would also be desirable to provide a method(s) for identifying subjects who are at risk for stroke through the detection of PDE4D splice-variant(s) that are associated with stroke.

### SUMMARY OF THE INVENTION

Accordingly, the present invention provides methods for identifying a subject at risk for stroke. In a particularly preferred embodiment of the present invention, a subject at risk for stroke may be identified by the detection of a PDE4D variant associated with stroke found in the subject. Exemplary PDE4D variants associated with stroke may be a polynucleotide or a polypeptide encoded by the polynucleotide. For the present invention, several PDE4D SNPs associated with stroke were identified in African-American and Caucasian females, providing support for association of this gene with stroke in non-Icelandic populations and among young adults. A method for identifying a subject at risk for stroke, in an alternative exemplary embodiment, includes the detection of a PDE4D variant in linkage disequilibrium with another allele that is associated with stroke. The secondary allele may be contained within the PDE4D gene.

Another particularly preferred embodiment of the present invention is a method of screening a subject for stroke susceptibility. The method includes detecting a risk allele within a sample from the subject, wherein the risk allele detected is in the PDE4D gene. The detection of the risk allele provides an indication of susceptibility to stroke based on the detection of the risk allele. The subject may be provided an indication of stroke susceptibility for ischemic stroke or a sub-type of ischemic stroke. The screening may include detecting a risk allele in the PDE4D gene that is in linkage disequilibrium with another allele that is associated with stroke. The secondary allele may be contained within the PDE4D gene.

In another particularly preferred embodiment of the present invention, a genetic marker which identifies a subject at risk for stroke is provided. The genetic marker is the PDE4D SNP rs918592. The SNP rs918592 site within the PDE4D gene may include either an “A” allele or “G” allele. The “A” allele confers a risk of stroke as compared to the “G” allele. Therefore, in an exemplary embodiment, the genetic marker is the “A” allele of the SNP rs918592.

In another particularly preferred embodiment of the present invention, a method for identifying a subject at risk for stroke includes identifying the presence of the “A” allele of the SNP rs918592. As stated above, the detection of the “A” allele identifies a risk of stroke as compared to the “G” allele. Through the utilization of factors from a statistical regression analysis model, association of an allele with stroke is shown by its Odds Ratio (OR) value and probability (p) value. The significant association of the rs918592 risk allele with stroke is shown by its OR and p values (OR=1.49, p=0.007). Wherein an OR value of 1 or greater indicates that the event is more likely to occur than not and wherein the lower/smaller the p value the higher the probability that the indices being measured did not show association by mere chance, but are the result of other non-probabilistic factors.

In a particularly preferred embodiment, a method for identifying a subject at risk for stroke includes the detection of the risk allele of SNP rs918592 in strong linkage disequilibrium (LD) with another allelic variant. The secondary allele may be contained within the PDE4D gene and have been previously associated with stroke in the Iceland population. Gretarsdottir S, Thorleifsson G, Reynisdottir S T, et al. The gene encoding phosphodiesterase 4D confers risk of ischemic stroke. Nat Genet 35: 131-138, 2003. These relationships are demonstrated in FIGS. 3 and 4 that demonstrate LD patterns and haplotype block structure among African-Americans and Caucasians, respectively.

In a particularly preferred embodiment of the present invention, a method of identifying a subject at risk for stroke includes detecting a risk allele within the PDE4D gene within the subject who is a smoker. In a still further particularly preferred embodiment of the present invention, a method identifying a subject at risk for stroke includes detecting an “A” allele of the SNP rs918592 within the subject who is a smoker. The current invention determined that the “A” allele confers a risk of stroke that appears to be mediated by smoking. As will be described below, the highly prevalent risk allele exhibited increased expression for smokers across ischemic stroke including all ischemic stroke subtypes.

In another particularly preferred embodiment of the present invention, a method for identifying a subject at risk for early onset stroke includes detecting a PDE4D allele associated with stroke in the subject who is between the ages of 15-49. Early onset stroke may be exhibited by a stroke event occurring in a subject who is between the ages of 15-49.

Another particularly preferred embodiment of the present invention is a method of screening a subject for stroke susceptibility. The method includes detecting a risk allele within a sample from the subject, wherein the risk allele detected is the “A” allele of the SNP rs918592. The detection of the risk allele provides an indication of stroke susceptibility within the subject. The subject may be provided an indication of stroke susceptibility for ischemic stroke or a sub-type of ischemic stroke.

It is a further particularly preferred embodiment of the present invention to provide a method for identifying a stroke associated genetic marker including obtaining a sample from a human subject, wherein the sample includes a PDE4D gene and identifying a risk allele within an SNP of the PDE4D gene having a p value less than or equal to 0.05 and an OR of 1 or more.

It is to be understood that both the foregoing general description of particularly preferred embodiments and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1 is an illustration of an association mapping of the PDE4D gene polymorphisms with ischemic stroke in the Stroke Prevention in Young Women (SPYW) study of the current invention. Age-adjusted P-values among the overall population (race adjusted), Caucasians and African-Americans for all SNPs analyzed using an additive model. The PDE4D gene structure is shown at the bottom. Solid boxes indicate the first exons for various isoforms, other exons are indicated by open boxes. Exons 2-5 are shown together, and exons 7-15 are shown together. Sideways arrows indicate promoters. Parenthesis indicate isoforms. The upward arrows on the gene demonstrate the SNP position within the gene as listed in Table 2;

FIG. 2 is a bar chart illustration of ORs and P-values for SNP rs918592 among never-, former-, all current-smokers and among light smokers (1-10 per day) and heavy smokers (11+per day) demonstrating a strong dose-response relationship;

FIG. 3 is a diagrammatic illustration of a Haplotype block structure among African-Americans. Arrows indicate SNPs rs918592 (associated with stroke in the SPYW2 population) and rs152312 (deCODE SNP 41, associated with stroke in the Iceland population) are in strong linkage disequilibrium as indicated by the circle;

FIG. 4 is a diagrammatic illustration of a Haplotype block structure among Caucasians;

FIG. 5 is an illustrative summary of PDE4D replication association studies in various populations. (A) The PDE4D gene structure. Exons are indicated by boxes. Differential promoters for PDE4D isoforms are indicated by horizontal arrows on the gene. The alternative splicing patterns of PDE4D isoforms are shown below the gene structure. The SNPs that were associated with stroke in the Icelandic population (Gretarsdottir S, Thorleifsson G, Reynisdottir S T, et al. The gene encoding phosphodiesterase 4D confers risk of ischemic stroke. Nat Genet 35: 131-138, 2003) are shown above the gene structure by the vertical arrows. (B) The SNP position throughout the PDE4D gene used in the replication studies. (C) A summary of these replication studies with asterisks (*) representing SNPs that reached statistical significance in association with stroke;

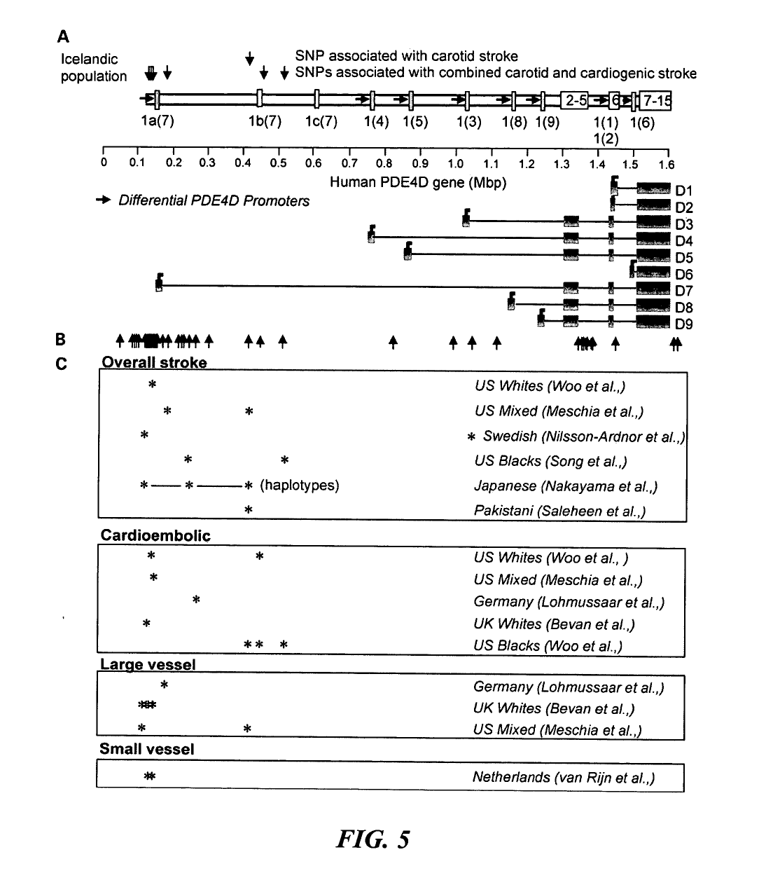

FIG. 6 is an illustration of a block diagram representation of a method of identifying a subject at risk for stroke in accordance with an exemplary embodiment of the present invention;

FIG. 7 is an illustration of a block diagram representation of a method of identifying a subject at risk for early-onset stroke in accordance with an exemplary embodiment of the present invention;

FIG. 8 is an illustration of a block diagram representation of a method of diagnosing a subject at risk for a particular type of stroke in accordance with an exemplary embodiment of the present invention; AND

FIG. 9 is an illustration of a block diagram representation of a method identifying a stroke associated genetic marker based on statistical analysis showing a p value less than or equal to 0.05 and an OR of 1 or more.

### DETAILED DESCRIPTION OF THE INVENTION

Reference will now be made in detail to the presently preferred embodiments of the invention, examples of which are illustrated in the accompanying drawings.

The present invention identifies and provides a novel allele (“risk allele” ) within the PDE4D gene that is associated with stroke. In a preferred embodiment, the risk allele is a single nucleotide polymorphism (SNP), within the PDE4D gene that is associated with stroke. As previously described, the association of the risk allele with stroke may be shown through various methods, for example, a statistical regression analysis model may be employed wherein an odds ratio (OR) value is equal to or greater than one (1) and a probability factor (p) is equal to or less than 0.05. The risk allele is useful as a genetic marker, wherein the expression within a subject provides an indication of the subject's susceptibility to stroke. In a more particularly preferred embodiment, the current invention includes a novel risk allele (genetic marker), SNP rs918592, which shows a significant association with stroke, particularly with ischemic stroke, heretofore unidentified. This significant association is exhibited by OR=1.49 and p=0.007 values for SNP rs918592. The SNP rs918592 site within the PDE4D gene may include either an “A” allele or “G” allele. The “A” allele confers a risk of stroke as compared to the “G” allele. Therefore, the novel genetic marker is the “A” allele of the SNP rs918592. The research methods and techniques employed in making these discoveries and determining their level of association with stroke are described below.

In a current embodiment of the present invention, various risk alleles of the PDE4D gene, such as SNP rs918592, may be used as a marker for identifying stroke susceptibility in subjects, such as multi-racial human females between the ages of 15-49. This SNP shows a strong association with stroke independently and further exhibits linkage disequilibrium (described below) with other alleles that show association with stroke. It is contemplated that the subjects may be other mammals, such as a human male and the like, and may have alternative age ranges, such as younger than 15 and older than 49 years of age. The presence of the marker may be detected in various manners, such as through obtaining a sample from the subject, wherein the sample includes the genetic risk factor (i.e., rs918592). The type and source of the sample for the subject is not critical and may be any material (e.g., cells, cell lines) that contains the subject's genetic information and the risk allele, for example, the sample may be from a hair follicle, urine, blood, serum, and the like. Various other sample sources and types as may be contemplated by those of ordinary skill in the art may also be employed by the present invention.

In a preferred embodiment, the subject characteristics including demographic and risk factor characteristics are shown and described in Table 1. In the current embodiment, these characteristics were gathered and analyzed in a case-control status. As stated previously, the ages of the subjects may range between 15 and 49. The mean age of the cases was 41.7 years and the mean age of control subjects was 39.6 years. Cases were significantly more likely to have a history of hypertension (p<0.0001), diabetes (p=0.0002), angina/MI (p=0.0005), to currently smoke cigarettes (p<0.0001), and to report the use of oral contraceptive pills (OCP) within the month (31 days) prior to their stroke (p=0.032). The subject characteristics described herein are exemplary and not intended to limit the scope of the characteristics which may be found in subjects. Therefore, the novel markers of the present invention may be found in subjects having alternative characteristics including one or more of the characteristics described and shown herein, or a combination of currently described and alternative characteristics.

TABLE 1
Characteristics by case-control status

| | Cases (n ¼ 224) | Controls (n ¼ 211) | P-value |
|---|---|---|---|
| Mean age (years) | 41.7 | 39.6 | 0.0026 |
| African-American (%) | 47.3 | 43.1 | 0.579 |
| Hypertension (%) | 41.1 | 14.2 | <0.0001 |
| Diabetes mellitus (%) | 17.9 | 6.2 | 0.0002 |
| Current smokers (%) | 47.8 | 23.7 | <0.0001 |
| Angina/MI (%) | 11.6 | 2.8 | 0.0005 |
| OCP (%)ₐ | 12.2 | 6.2 | 0.032 |

*Two cases and one control could not recall their last OCP use, therefore cases n ¼ 222 and controls n ¼ 210.

It is contemplated that stroke associated variants of the PDE4D gene may be variously positioned within the gene. The stroke associated variant rs918592 is positioned in the non-coding conserved segments of the PDE4D gene, generally located at the 5′ end of the gene. More particularly, the risk allele of SNP rs918592 of the present invention is within an intron(s) located at the 5′ end of the PDE4D gene. It is contemplated that the regions around the first exons at the 5′ end of the PDE4D gene and various other regions throughout the PDE4D gene may include multiple variants that may exhibit association with stroke and/or linkage disequilibrium (“LD”) with the SNP rs918592 of the present invention and provide further stroke association characteristics, as will be described below. A total of 71 polymorphisms (66 SNPs and 5 insertion/deletion polymorphisms) are identified by the present invention. Of these, 41 have a minor allele frequency ≧0.05 in at least one population. Of these 41 polymorphisms, 19 were in African-Americans only, 4 in Caucasians only, and 18 in both races. Many of these SNPs are not listed in public or private databases (National Center for Biotechnology Information (NCBI), Celera's Human SNP Reference Database, The SNP Consortium, Human Genome Variation Database, and others). Table 2 lists the genotyped SNPs as ordered by their physical position within the gene. Table 2 further discloses the allelic variants and minor allele frequencies among cases and controls stratified by race. These SNPs are located throughout the entire gene. All genotyped SNPs were in Hardy-Weinberg equilibrium.

TABLE 2
SNPs analyzed in complete case - control study population including allelic variants, position,
genotype call rate and minor allele frequencies as stratified by race and case/control status

| | | | Allele frequency^{b}/n | | | | | |
|---|---|---|---|---|---|---|---|---|
| | | | African-American | | | Caucasians | | |
| SNP name or rs number, alleles | Position^{a} | Call rate | Cases | Controls | P-value^{c} | Cases | Controls | P-value^{c} |
| PDE4D-32913 (Novel), C/T^{d} | 32913 | 85 | 0.235/78 | 0.272/76 | 0.343 | 0.589/82 | 0.568/74 | 0.918 |
| rs152312 (Decode 41), C/T | 144510 | 99 | 0.028/102 | 0.044/88 | 0.246 | 0.133/93 | 0.085/99 | 0.259 |
| rs153031 (Decode 42), C/T^{d} | 145035 | 93 | 0.353/75 | 0.338/65 | 0.851 | 0.607/75 | 0.657/70 | 0.413 |
| rs12188950 (Decode 45), C/T | 149009 | 99 | 0.129/101 | 0.137/89 | 0.750 | 0.163/94 | 0.116/98 | 0.207 |
| rs27224, G/T | 154835 | 99 | 0.033/102 | 0.050/89 | 0.293 | 0.133/93 | 0.085/99 | 0.259 |
| rs153067, C/T^{d} | 168927 | 99 | 0.355/83 | 0.358/71 | 0.555 | 0.612/76 | 0.669/74 | 0.322 |
| rs42222, A/C | 216712 | 99 | 0.043/101 | 0.079/87 | 0.186 | 0.104/90 | 0.066/98 | 0.366 |
| PDE4D-229902 (Novel), G/A^{d} | 229902 | 97 | 0.245/100 | 0.170/86 | 0.130 | 0.011/95 | 0.005/86 | 0.976 |
| rs918590, G/T | 234518 | 95 | 0.462/99 | 0.463/83 | 0.833 | 0.246/91 | 0.200/98 | 0.264 |
| rs918592, A/G | 235023 | 97 | 0.340/102 | 0.455/89 | 0.056 | 0.750/94 | 0.825/99 | 0.100 |
| PDE4D-249037 (Novel), C/T^{d} | 249037 | 90 | 0.282/75 | 0.271/69 | 0.615 | 0.040/75 | 0.052/67 | 0.706 |
| PDE4D-327964 (Novel), A/G^{d} | 327964 | 93 | 0.247/94 | 0.222/85 | 0.542 | 0.006/85 | 0.016/90 | 0.467 |
| rs966221(Decode 83), C/T | 429806 | 95 | 0.495/101 | 0.489/88 | 0.990 | 0.608/91 | 0.525/98 | 0.078 |
| rs1396476 (Decode 89), G/T | 535684 | 99 | 0.152/101 | 0.073/88 | 0.034 | 0.145/92 | 0.157/98 | 0.716 |
| PDE4D-866896 (Novel), G/A^{d} | 866896 | 95 | 0.026/76 | 0.044/65 | 0.273 | 0.207/75 | 0.225/71 | 0.371 |
| PDE4D-1024373 (Novel), A/G^{d} | 1024373 | 79 | 0.119/62 | 0.161/53 | 0.410 | 0.083/66 | 0.070/64 | 0.676 |
| rs294496, C/T^{d} | 1053899 | 78 | 0.213/78 | 0.261/68 | 0.337 | 0.233/73 | 0.187/74 | 0.303 |
| rs525099, C/T^{d} | 1242015 | 78 | 0.187/76 | 0.201/66 | 0.924 | 0.336/74 | 0.395/80 | 0.349 |
| PDE4D-1483844 (Novel), C/T | 1483844 | 83 | 0.020/97 | 0.023/83 | 0.748 | 0.051/88 | 0.098/91 | 0.133 |
| PDE4D-1483907 (Novel), A/G^{d} | 1483907 | 84 | 0.121/86 | 0.111/78 | 0.703 | 0.218/78 | 0.195/81 | 0.765 |
| rs929820, C/T^{d} | 1538994 | 95 | 0.380/96 | 0.447/83 | 0.721 | 0.710/87 | 0.643/97 | 0.129 |
| PDE4D-1568979 (Novel), C/T^{d} | 1568979 | 94 | 0.064/98 | 0.077/82 | 0.908 | 0.006/83 | 0/93 | 0.990 |
| rs1498606, C/T | 1594777 | 95 | 0.257/99 | 0.202/87 | 0.908 | 0.176/90 | 0.116/98 | 0.189 |

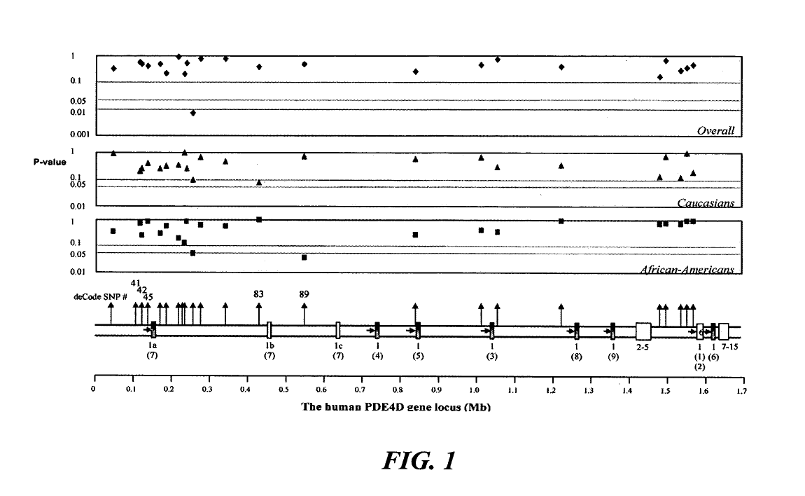

^{a}Relative position (148436-148557 exon 1a) (7).
^{b}As listed by minor allele frequency in African-Americans (bolded in column 1).
^{c}In race-stratified age-adjusted additive model, (primary analyses) as denoted in FIG. 1.
^{d}SNP genotyped using the UHT system.

Utilizing various modeling techniques, the present invention describes the association to stroke exhibited by the novel risk allele. In a preferred embodiment, the present invention utilized a minimally-adjusted model. FIG. 1 illustrates the 23 analyzed SNPs, shown above in Table 2, denoting their position within PDE4D. Initial levels of significance obtained by comparing allele frequencies between cases and controls, are shown for the entire population and for African-American and Caucasian women separately. Through the minimally-adjusted model, using an additive model with adjustment for age and race, the “A” allele of SNP rs9185982 has been identified as being significantly associated with stroke (OR=1.49, p=0.007). Significant association with stroke, under the statistical regression analysis used (described below), may be shown in various SNPs of genes through identification of particular values, such as OR ≧1 and p ≦0.05. It is contemplated that various OR and p values may be shown to associate a particular SNP and allelic variant thereof with stroke without departing from the scope and spirit of the present invention. The risk allele of rs918592 (the “A” allele) showed a similar degree of association among both races (African American OR=1.50, p=0.056 and Caucasian OR 1.52, p=0.010). The “A” allele occurred more frequently in African Americans (frequencies among cases and controls=0.66 and 0.55, respectively) than in Caucasians (frequencies among cases and controls=0.25 and 0.18, respectively). The significant association of the risk allele with stroke and the similarity in degree of association for rs918592 between different ethnicities is detailed in Tables 3 and 4 (below). It is contemplated that multiple alternative risk alleles with the PDE4D gene may be associated with stroke utilizing similar or alternative modeling techniques. For example, other SNPs associated with stroke are also listed in Table 3. Allele frequencies and analyses for SNP variants within the PDE4D gene are shown in Table 2.

TABLE 3
Crude and adjusted odds ratios, p-values, and 95% confidence intervals for SNPs found to be significantly
associated with stroke within the crude or vascular model. Significant results (p < 0.05) bolded.

| | | | | | | | | Vascular Model | | | |
|---|---|---|---|---|---|---|---|---|---|---|---|
| | | | | | | | | (age, smoking, OCP, | | | |
| SNP/Risk | Inheritance | Controls/ | | Age adjusted | | | | HTN, DM, angina/MI) | | | |
| Allele | Model | Cases** | Race | OR | 95% CI | | P | OR | 95% CI | | P |
| rs918592/A | Dominant | 207/219 | ALL* | 1.84 | 1.17 | 2.88 | 0.009 | 1.62 | 0.99 | 2.66 | 0.054 |
| | | 89/102 | Afr-Amrn | 2.44 | 1.10 | 5.42 | 0.029 | 2.34 | 0.98 | 5.57 | 0.055 |
| | | 99/94 | Caucasian | 1.70 | 0.94 | 3.08 | 0.080 | 1.34 | 0.69 | 2.62 | 0.393 |
| rs153031/C | Dominant | 151/169 | ALL* | 1.71 | 0.99 | 2.94 | 0.055 | 1.50 | 0.81 | 2.78 | 0.201 |
| | | 65/75 | Afr-Amrn | 1.77 | 0.63 | 5.01 | 0.282 | 4.31 | 1.10 | 16.92 | 0.036 |
| | | 70/75 | Caucasian | 1.52 | 0.78 | 2.96 | 0.221 | 1.08 | 0.50 | 2.34 | 0.849 |
| rs966221/T | Additive | 205/214 | ALL* | 1.17 | 0.89 | 1.54 | 0.266 | 1.21 | 0.90 | 1.64 | 0.198 |
| | | 88/101 | Afr-Amrn | 1.00 | 0.64 | 1.50 | 0.990 | 0.95 | 0.61 | 1.49 | 0.831 |
| | | 98/91 | Caucasian | 1.44 | 0.96 | 2.15 | 0.078 | 1.74 | 1.10 | 2.75 | 0.019 |
| rs1396476/G | Additive | 205/215 | ALL* | 1.14 | 0.77 | 1.72 | 0.512 | 1.20 | 0.77 | 1.87 | 0.422 |
| | | 88/101 | Afr-Amrn | 2.19 | 1.06 | 4.55 | 0.034 | 2.67 | 1.19 | 5.99 | 0.017 |
| | | 98/92 | Caucasian | 0.90 | 0.51 | 1.58 | 0.716 | 1.01 | 0.53 | 1.89 | 0.989 |
| rs1498606/T | Recessive | 204/212 | ALL* | 1.26 | 0.83 | 1.91 | 0.279 | 1.34 | 0.86 | 2.13 | 0.223 |
| | | 87/99 | Afr-Amrn | 1.03 | 0.56 | 1.89 | 0.931 | 0.96 | 0.49 | 1.86 | 0.907 |
| | | 98/90 | Caucasian | 1.66 | 0.86 | 3.22 | 0.132 | 2.17 | 1.02 | 4.63 | 0.044 |

*Includes Hispanics, Asian, and all others. Adjusted for age, and race (black, white, and other).
**Indicates number of controls and cases used in crude analyses.
NC - indicates insufficient sample size to perform calculation; model did not converge

TABLE 4
SNP rs918592-additive model

| | | | | | | Vascular model | | | |
|---|---|---|---|---|---|---|---|---|---|
| | | | | | | (age, smoking, OCP, | | | |
| | | Age-adjusted | | | | HTN, DM, angina/MI) | | | |
| Race | Controls/cases^{a} | OR | 95% CI | | P-value | OR | 95% CI | | P-value |
| All^{b} | 207/219 | 1.49 | 1.11 | 2.00 | 0.007 | 1.38 | 0.99 | 1.90 | 0.052 |
| African-American | 89/102 | 1.50 | 0.99 | 2.26 | 0.029 | 1.40 | 0.89 | 2.20 | 0.146 |
| Caucasian | 99/94 | 1.52 | 0.93 | 2.48 | 0.080 | 1.35 | 0.78 | 2.31 | 0.284 |

Age-adjusted and vascular models adjusted ORs, 95% confidence intervals and P-values. Significant results (P, 0.05) are in bold.
^{a}Indicates number of controls and cases used in crude analyses.
^{b}Includes Hispanics, Asian and all others. Adjusted for race (black, white and others).

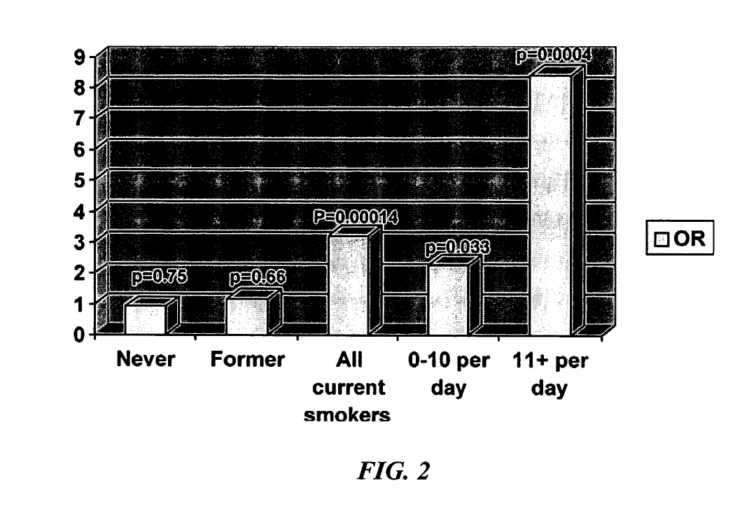

As shown in FIG. 2, there is a gene X environment interaction (p=0.03); the effect of this was the identification that SNP rs918592 was highly associated with stroke among smokers (OR=3.22, p<0.00014), showing a decreased association with stroke among never-smokers (OR=0.93, p=0.75) or former smokers (OR=1.16, p=0.66). Cigarette smoking causes endothelial dysfunction (Celermajer D S, Sorensen K, Georgakopoulis D, et al. Cigarette smoking is associated with dose-related and potentially reversible impairment of endothelium-dependent dilatation in healthy young adults. Circulation. 1993; 88: 2149-2155) and is known to modify the expression of many genes in endothelial cells, including cAMP response element binding protein (CREB). (Zhang S, Day I N, Ye S. Microarray analysis of nicotine-induced changes in gene expression in endothelial cells. Physiol Genomics. Apr. 27, 2001; 5(4):187-92.) CREB is a transcription factor that induces the expression of a panel of genes with established roles in cell survival, metabolism, and plasticity in the nervous system, including a potential role in ischemic preconditioning. (Ratan R R. cAMP response element binding protein family transcription factors: the Holy Grail of neurological therapeutics? Ann Neurol. 2004 November; 56(5):607-8.) Thus, both PDE4D and smoking may mediate effects via CREB providing a potential mechanism for the smoking interaction. It is contemplated that other mechanisms of action that result in the expression of the risk allele of the PDE4D gene within a subject may occur without departing from the scope and spirit of the present invention. An alternative mechanism may be that smoking may potentiate the effect of increased PDE4D expression. In a mouse model, prenatal exposure to cigarette smoking increased PDE4D messenger RNA expression and decreased cAMP in the lung. (Singh S P, Barrett E G, Kalra R, et al. Prenatal cigarette smoke decreases lung cAMP and increases airway hyperresponsiveness. Am J Respir Crit Care Med. Aug. 1, 2003; 168(3):342-7).

Table 5 shows that SNP rs918592 is highly associated with stroke among smokers (OR=3.22, p=0.00014). A decreased association with stroke among never-smokers (OR=0.93, p=0.75) or former smokers (OR=1.16, p=0.66) is also shown. Through the use of both an environmental and an environment/vascular model (described below) risks among all participants (Caucasian, African-American, and all other) for SNP rs918592, as stratified by age and other stroke risk factors with associated adjusted odds ratios, p-values, and 95% confidence intervals, are identified and provided.

TABLE 5
Among all participants, SNP rs918592-additive model, stratified by
age and other stroke risk factors demonstrating adjusted ORs,
95% confidence intervals and P-values
rs918592-additive

| Risk factor^{a} | Strata | Controls/cases | OR^{a} | 95% | CI | P-value |
|---|---|---|---|---|---|---|
| Age | 14-39 | 79/57 | 0.95 | 0.54 | 1.67 | 0.871 |
| | 40-49 | 128/162 | 1.85 | 1.29 | 2.65 | 0.001 |
| OCP use | − | 193/191 | 1.55 | 1.14 | 2.10 | 0.005 |
| | + | 13/26 | 1.20 | 0.33 | 4.35 | 0.787 |
| Smoking | Never | 115/82 | 0.93 | 0.60 | 1.46 | 0.752 |
| | Former | 44/36 | 1.16 | 0.61 | 2.21 | 0.658 |
| | Current | 48/101 | 3.22 | 1.76 | 5.87 | 0.00014 |
| HTN | − | 178/127 | 1.79 | 1.24 | 2.57 | 0.002 |
| | + | 29/92 | 0.90 | 0.49 | 1.64 | 0.714 |
| DM | − | 193/181 | 1.57 | 1.14 | 2.15 | 0.005 |
| | + | 14/38 | 0.71 | 0.25 | 2.00 | 0.513 |
| Angina/MI | − | 202/195 | 1.53 | 1.13 | 2.08 | 0.006 |
| | + | 5/24 | NC | NC | NC | NC |

Significant results (P, 0.05) are in bold. NC indicates insufficient sample size to perform calculation; model did not converge.
^{a}Each stroke risk factor adjusted for age and race (black, white and others).

As shown in FIG. 2, the smoking by genotype interaction term was significant (p=0.03) in a model including the covariates of age, race, hypertension, diabetes mellitus, and oral contraceptive. The risk genotype showed a decreased association with any of the stroke risk factors. Thus, rs918592 exhibits a dose dependent relationship for subjects who are smokers.

The remaining 22 SNPs, from FIG. 1 and Table 2, were evaluated using these models and several additional SNPs were associated with stroke, including: rs966221 (SNP 83), rs1396476 (SNP 89), and rs1501647 under an additive model; rs153031 (SNP 42) and rs918592 under a dominant model; and rs1498606 under a recessive model.

Details regarding these results are provided in Table 3 (above). Risk factor stratified analyses of these associated SNPs are provided in Table 6.

TABLE 6
Among all participants, significant SNPs stratified by age and other stroke risk factors
with associated adjusted odds ratios, p-values, and 95% confidence intervals.
Significant results (p < 0.05) bolded.

| SNP - | | | | | | | |
|---|---|---|---|---|---|---|---|
| Model | Risk factor | Strata | Controls/Cases | OR* | 95% CI | | P |
| rs918592 - | AGE | 14-39 | 79/57 | 0.74 | 0.34 | 1.62 | 0.445 |
| Dominant | | 40-49 | 128/162 | 2.98 | 1.69 | 5.28 | 0.0002 |
| | OC | − | 193/191 | 1.98 | 1.22 | 3.21 | 0.006 |
| | | + | 13/26 | 0.72 | 0.16 | 3.23 | 0.665 |
| | Smoking | Never | 115/82 | 0.87 | 0.33 | 2.29 | 0.781 |
| | | Former | 44/36 | 1.09 | 0.41 | 2.91 | 0.858 |
| | | Current | 48/101 | 4.29 | 1.81 | 10.19 | 0.0001 |
| | HTN | − | 178/127 | 2.37 | 1.38 | 4.09 | 0.002 |
| | | + | 29/92 | 0.84 | 0.30 | 2.38 | 0.741 |
| | DM | − | 193/181 | 1.99 | 1.24 | 3.19 | 0.005 |
| | | + | 14/38 | 0.26 | 0.03 | 2.55 | 0.249 |
| | Angina/MI | − | 202/195 | 1.99 | 1.25 | 3.18 | 0.004 |
| | | + | 5/24 | NC | NC | NC | NC |
| rs153031 - | AGE | 14-39 | 54/49 | 0.87 | 0.35 | 2.17 | 0.758 |
| Dominant | | 40-49 | 97/120 | 2.55 | 1.28 | 5.12 | 0.008 |
| | OC | − | 140/143 | 1.82 | 1.01 | 3.29 | 0.046 |
| | | + | 10/24 | 0.91 | 0.14 | 6.10 | 0.922 |
| | Smoking | Never | 87/59 | 1.41 | 0.63 | 3.19 | 0.409 |
| | | Former | 33/30 | 1.94 | 0.52 | 7.25 | 0.323 |
| | | Current | 31/80 | 2.11 | 0.76 | 5.80 | 0.150 |
| | HTN | − | 128/103 | 1.96 | 1.06 | 3.63 | 0.033 |
| | | + | 23/66 | 0.36 | 0.04 | 3.26 | 0.360 |
| | DM | − | 141/140 | 2.03 | 1.12 | 3.68 | 0.020 |
| | | + | 10/29 | 0.70 | 0.11 | 4.29 | 0.695 |
| | Angina/MI | − | 148/147 | 1.98 | 1.12 | 3.54 | 0.020 |
| | | + | 3/22 | NC | NC | NC | NC |
| rs966221 - | AGE | 14-39 | 77/56 | 1.62 | 0.94 | 2.77 | 0.081 |
| Additive | | 40-49 | 128/158 | 0.98 | 0.71 | 1.36 | 0.982 |
| | OC | − | 191/186 | 1.19 | 0.74 | 1.93 | 0.470 |
| | | + | 13/26 | 1.96 | 0.68 | 5.63 | 0.211 |
| | Smoking | Never | 114/80 | 1.21 | 0.80 | 1.86 | 0.362 |
| | | Former | 44/34 | 2.17 | 1.10 | 4.26 | 0.025 |
| | | Current | 47/100 | 0.87 | 0.53 | 1.42 | 0.572 |
| | HTN | − | 176/126 | 1.20 | 0.86 | 1.67 | 0.285 |
| | | + | 29/88 | 1.20 | 0.67 | 2.15 | 0.541 |
| | DM | − | 191/178 | 1.18 | 0.88 | 1.57 | 0.272 |
| | | + | 14/36 | 1.23 | 0.48 | 3.13 | 0.668 |
| | Angina/MI | − | 200/190 | 1.22 | 0.76 | 1.06 | 0.413 |
| | | + | 5/24 | NC | NC | NC | NC |
| rs1396476 - | AGE | 14-39 | 79/55 | 0.60 | 0.28 | 1.29 | 0.194 |
| Additive | | 40-49 | 126/160 | 1.61 | 0.95 | 2.72 | 0.076 |
| | OC | − | 191/187 | 1.29 | 0.83 | 1.99 | 0.255 |
| | | + | 13/26 | 0.43 | 0.11 | 1.65 | 0.215 |
| | Smoking | Never | 115/80 | 0.98 | 0.98 | 1.77 | 0.940 |
| | | Former | 44/35 | 1.06 | 0.41 | 2.73 | 0.904 |
| | | Current | 46/106 | 1.60 | 0.72 | 3.56 | 0.253 |
| | HTN | − | 177/124 | 1.01 | 0.63 | 1.61 | 0.981 |
| | | + | 28/91 | 2.24 | 0.72 | 7.00 | 0.163 |
| | DM | − | 191/178 | 1.13 | 0.74 | 1.71 | 0.574 |
| | | + | 14/37 | 3.16 | 0.33 | 3.03 | 0.321 |
| | Angina/MI | − | 200/191 | 1.17 | 0.78 | 1.77 | 0.452 |
| | | + | 5/24 | NC | NC | NC | NC |
| rs1498606 - | AGE | 14-39 | 79/54 | 2.25 | 1.05 | 4.81 | 0.036 |
| Recessive | | 40-49 | 125/158 | 0.96 | 0.58 | 1.60 | 0.888 |
| | OC | − | 190/184 | 1.26 | 0.81 | 1.98 | 0.307 |
| | | + | 13/26 | 0.49 | 0.11 | 2.22 | 0.351 |
| | Smoking | Never | 113/79 | 1.98 | 1.08 | 3.66 | 0.028 |
| | | Former | 44/36 | 0.61 | 0.23 | 1.66 | 0.332 |
| | | Current | 47/97 | 1.05 | 0.48 | 2.33 | 0.901 |
| | HTN | − | 176/122 | 1.12 | 0.68 | 1.84 | 0.654 |
| | | + | 28/90 | 2.14 | 0.79 | 5.85 | 0.136 |
| | DM | − | 190/175 | 1.36 | 0.87 | 2.11 | 0.173 |
| | | + | 14/37 | 0.92 | 0.22 | 3.76 | 0.902 |
| | Angina/MI | − | 199/189 | 1.22 | 0.80 | 1.89 | 0.360 |
| | | + | 5/23 | NC | NC | NC | NC |

*Each stroke risk factor adjusted for age and race (black, white, and other).
NC - indicates insufficient sample size to perform calculation, model did not converge.

In contrast to the findings from Iceland (Gretarsdottir S, Thorleifsson G, Reynisdottir S T, et al. The gene encoding phosphodiesterase 4D confers risk of ischemic stroke. Nat Genet 35: 131-138, 2003) and the recent American study (Meschia J F, Brott T G, Brown R D Jr, Crook R, Worrall B B, Kissela B, Brown W M, Rich S S, Case L D, Evans E W, Hague S, Singleton A, Hardy J. Phosphodiesterase 4D and 5-lipoxygenase activating protein in ischemic stroke. Ann Neurol. 2005 September; 58(3):351-61), the strongly associated SNP rs918592, of the current invention, was associated with multiple ischemic stroke subtypes. The current invention also discloses ischemic stroke subtype association with various risk alleles within the PDE4D gene and that risk alleles within the PDE4D gene, and more particularly the risk allele A of rs918592, have an effect of the PDE4D locus on stroke risk among young adults, a population with a very low prevalence of atherosclerotic disease. The findings of significant associations in a young stroke population within several subtypes including small vessel strokes and large vessel strokes is of novel significance which may assist in the prevention, screening for, diagnosis and/or treatment of stroke within this subpopulation. It is of further significance that the association of the various risk alleles of the PDE4D gene, particularly risk allele A of rs918592, may provide determinative proof of susceptibility with or without atherosclerosis which suggests that atherogenesis is unlikely to mediate the association.

Table 7 discloses the association of SNP rs918592 with various stroke subtypes. The results presented in Table 7 are stratified by stroke subtype with associated adjusted odds ratios, p-values, and 95% confidence intervals. Similar analyses regarding the other associated SNPs are shown in Table 8. The atherosclerotic group included 27 cases with either probable or possible atherosclerotic mechanism, the cardiac group included 14 cases with a probable cardiac source of embolism; the probable dissection group included 13 cases confirmed by neuroimaging; the lacunar group included 43 cases of symptomatic small deep lesions on neuroimaging studies or classic lacunar syndromes regardless of other potential causes; and the probable hematologic group included 9 cases. These categories may not be mutually exclusive. There were 125 non-lacunar stroke cases of undetermined etiology. SNP rs918592 was significantly associated with all stroke subtypes, but showed a decreased association with the cardiac and hematologic subtypes. Therefore, in a preferred embodiment of the present invention, rs918592 is a novel genetic marker for the identification of the susceptibility of a subject to various stroke sub-types. It is further contemplated that this novel genetic marker may be identified in LD with one or more secondary alleles. The secondary alleles may show some association with stroke and may be located within the PDE4D gene. The identification of the association of rs918592 with stroke provides a significant advantage over previous stroke associated allelic variants, as described above, which did not show association or exhibited weak association to various stroke sub-types.

TABLE 7
Among all participants, SNP rs918582-additive
model, stratified by stroke subtype demonstrating adjusted
ORs, 95% confidence intervals and P-values

| rs918592-additive | | | | | |
|---|---|---|---|---|---|
| Stroke subtype | Controls/cases | OR^{a} | 95% | CI | P |
| Atherosclerotic | 207/27 | 2.35 | 1.22 | 4.53 | 0.011 |
| Cardiac | 207/14 | 0.94 | 0.42 | 2.08 | 0.870 |
| Dissection | 207/13 | NC | NC | NC | NC |
| Lacunar | 207/45 | 1.85 | 1.12 | 3.05 | 0.017 |
| Hematologic | 207/9 | 0.21 | 0.04 | 1.10 | 0.065 |
| Non-lacunar, | 207/125 | 1.50 | 1.07 | 2.12 | 0.020 |
| undetermined | | | | | |

Significant results (P, 0.05) are in bold.
NC indicates insufficient sample size to perform calculation; model did not converge.
^{a}Adjusted for age and race (black, white and other).

TABLE 8
Among all participants, significant SNPs stratified by stroke subtype with
associated adjusted odds ratios, p-values, and 95% confidence intervals.
Significant results (P < 0.05) bolded.

| SNP - model | Stroke Subtype | Controls/Cases | OR* | 95% CI | | P |
|---|---|---|---|---|---|---|
| rs918592 - Dominant | Atherosclerotic | 207/27 | 5.40 | 1.72 | 16.92 | 0.004 |
| | Cardiac | 207/14 | 0.89 | 0.26 | 3.059 | 0.852 |
| | Dissection | 207/13 | NC | NC | NC | NC |
| | Lacunar | 207/45 | 3.15 | 1.329 | 7.462 | 0.009 |
| | Hematologic | 207/9 | 0.19 | 0.03 | 1.187 | 0.076 |
| | Non-Lacunar, undetermined | 207/121 | 1.64 | 0.99 | 2.862 | 0.053 |
| rs153031 - Dominant | Atherosclerotic | 151/21 | 1.79 | 0.54 | 5.91 | 0.338 |
| | Cardiac | 151/11 | 0.57 | 0.15 | 2.25 | 0.424 |
| | Dissection | 151/8 | NC | NC | NC | NC |
| | Lacunar | 151/31 | 2.05 | 0.66 | 6.37 | 0.216 |
| | Hematologic | 151/7 | NC | NC | NC | NC |
| | Non-Lacunar, undetermined | 151/96 | 1.85 | 0.97 | 3.53 | 0.061 |
| rs966221 - Additive | Atherosclerotic | 205/26 | 1.48 | 0.80 | 2.75 | 0.209 |
| | Cardiac | 205/14 | 2.44 | 1.04 | 5.72 | 0.040 |
| | Dissection | 205/13 | NC | NC | NC | NC |
| | Lacunar | 205/44 | 1.24 | 0.77 | 2.00 | 0.378 |
| | Hematologic | 205/8 | 2.96 | 0.91 | 9.67 | 0.072 |
| | Non-Lacunar, undetermined | 205/119 | 1.01 | 0.73 | 1.38 | 0.970 |
| rs1396476 - Additive | Atherosclerotic | 205/27 | 0.96 | 0.38 | 2.41 | 0.924 |
| | Cardiac | 205/14 | 0.97 | 0.29 | 3.26 | 0.962 |
| | Dissection | 205/13 | NC | NC | NC | NC |
| | Lacunar | 205/45 | 1.45 | 0.71 | 2.99 | 0.307 |
| | Hematologic | 205/8 | 0.42 | 0.06 | 3.02 | 0.391 |
| | Non-Lacunar, undetermined | 205/118 | 1.22 | 0.78 | 1.92 | 0.390 |
| rs1498606 - Recessive | Atherosclerotic | 204/27 | 0.62 | 0.23 | 1.67 | 0.343 |
| | Cardiac | 204/14 | 3.97 | 1.24 | 12.66 | 0.020 |
| | Dissection | 204/11 | NC | NC | NC | NC |
| | Lacunar | 204/45 | 1.04 | 0.49 | 2.21 | 0.910 |
| | Hematologic | 204/9 | 0.36 | 0.043 | 3.10 | 0.354 |
| | Non-Lacunar, undetermined | 204/116 | 1.52 | 0.94 | 2.46 | 0.088 |

*Adjusted for age and race (black, white, and other).
NC - indicates insufficient sample size to perform calculation, model did not converge.

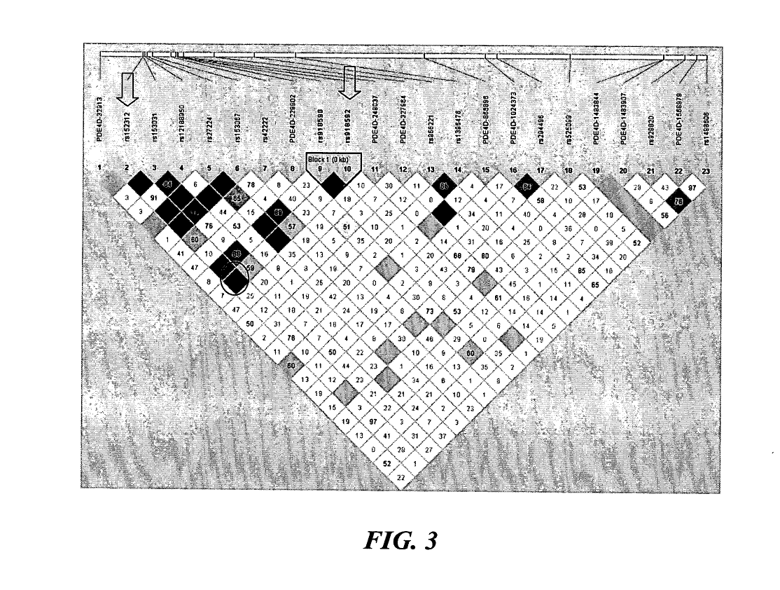

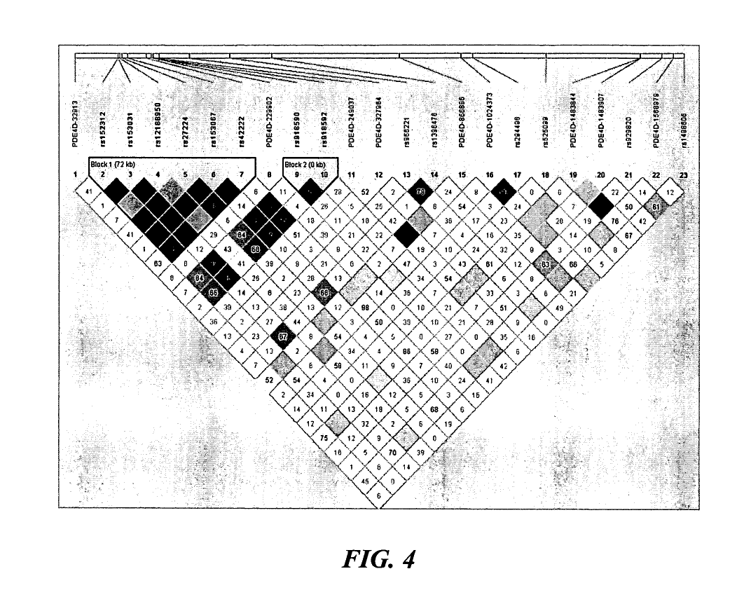

FIGS. 3 and 4 demonstrate the haplotype block structure among human females of African-American and Caucasian ethnicity, respectively. FIG. 4 shows the SNPs examined by all published studies of PDE4D and stroke, demonstrating that previous work has focused on the small portion of the gene where deCODE associations were found. Within both races, two (2) haplotype blocks (“Block 1” and “Block 2” ) are seen in the promoter 1 a (see FIG. 1) isoform region of the gene, with SNPs in strong linkage disequilibrium within and between blocks. In both races, the haplotype blocks are separated by PDE4D-229902. Without the presence of this intervening SNP these blocks might have been considered a single larger block, particularly given that the SNPs within each block are in linkage disequilibrium within and between blocks. Among African-Americans, the region of interest is between SNP rs152312 (SNP 41) and rs918592. Within Caucasians, the region of interest is bounded by SNP rs12188950 and rs918592. In both the African-American and Caucasian populations SNP rs918592, which is associated with stroke in the SPYW2 population, is in strong linkage disequilibrium with rs152312 (SNP 41) which is associated with stroke in the Iceland population (see FIGS. 3 and 4).

It is further shown in FIG. 4, that various risk alleles within the PDE4D gene are in LD with rs918592 and one another. The LD of these various risk alleles indicates that the identification of stroke association of allelic variants within the PDE4D gene may be accomplished through detection of the individual risk alleles and through the detection of multiple risk alleles in LD with one another. It is contemplated that the detection of “linked” allelic variants within the PDE4D gene may exhibit an increased or decreased stroke association. For example, the “linked” variant detected may exhibit an OR significantly higher than one (1) and the p value may be significantly lower than 0.05.

SNPs were identified throughout the entire PDE4D gene, particularly on the regions around the first exons at the 5′ end of the gene. Among the stroke-associated variants evaluated herein, several SNPs were found to be associated with ischemic stroke including: SNP 83(rs966221) among Caucasians in an additive model; SNP 89(rs1396476) among African-Americans using an additive model; and SNP 42(rs153031) in African-Americans using a dominant model. Additionally, SNP rs918592 of the current invention is found to be the most associated with stroke and is linkage disequilibrium with rs152312 (SNP 41), one of the polymorphisms more strongly associated with stroke in the Iceland population. Furthermore, the risk haplotype in the Iceland study contained rs152312 and had lower expression of the PDE4D7 isoform. (Gretarsdottir S, Thorleifsson G, Reynisdottir S T, et al. The gene encoding phosphodiesterase 4D confers risk of ischemic stroke. Nat Genet 35: 131-138, 2003.) The strongest association with ischemic stroke in the current study was in the 5′ region of PDE4D, near the first alternative exon for PDE4D7. Therefore, it is contemplated that the various risk alleles of the PDE4D gene may exhibit an increased or decreased association with stroke when compared with rs918592.

The present invention includes methods for identifying subjects susceptible to stroke through detection of SNPs which contain risk alleles within the PDE4D gene. The methods of the present invention may be employed at various times relative to a stroke event, which is a stroke experienced by a subject. For example, the method may be employed with a subject before an initial stroke event has been experienced by the subject. Alternatively, the method may be employed in a secondary environment, where it is employed after a stroke event has been experienced by the subject.

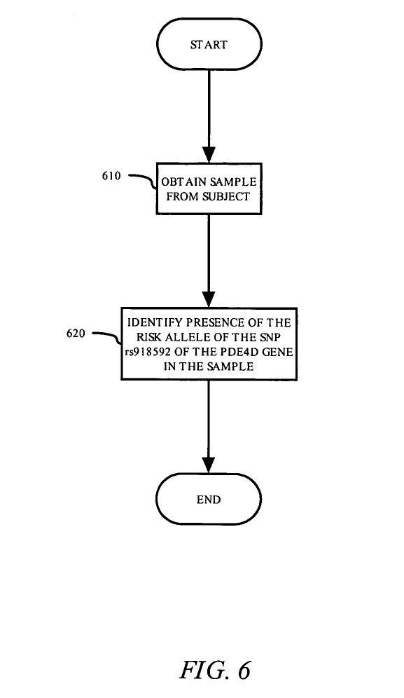

In a preferred embodiment, shown in FIG. 6, a method of identifying a subject at risk for stroke includes a first step 610 of obtaining a sample from a subject. In a second step 620, a risk allele of the SNP rs918592 which is associated with stroke is detected in the sample. Upon detection of this risk allele the method may further comprise the step of providing preventative and/or treatment options to the subject. In an alternative embodiment, the second step of the method may be the detection of a risk allele from the PDE4D gene which is associated with stroke.

Thus, the present invention contemplates a genetic test that may be used to identify subjects with a susceptibility to stroke through the detection of a risk allele within the PDE4D gene, such as the A allele of rs918592. The test may include the detection of a risk allele from the PDE4D gene within a sample. Further, the present invention contemplates providing counseling to subjects identified as having the risk factor. The counseling may include guidance to quit smoking or not to start smoking as this may be a critical factor in increasing a subject's risk for stroke.

As previously stated, various subjects may be used and the sample may be of various types and obtained from various sources. Therefore, it is contemplated that the subject is a human, female or male, and within an age range of 15-49 or the subject may be older than 49 or younger than 15. Further, the subject may be a smoker, heavy or light, non-smoker, or never smoker. The subject may have a pre-existing condition or be consuming various pharmacologically active products, as previously identified.

In another preferred embodiment, a method for identifying a subject at risk for stroke includes the step of detecting a risk allele of the PDE4D gene associated with stroke, the risk allele being in linkage disequilibrium (LD) with another (secondary) allele that is associated with stroke. For example, the risk allele expressed in rs918592 may be detected in linkage disequilibrium with rs152312 (SNP 41) that has been associated with stroke. It is contemplated that the risk allele of the PDE4D gene, including the risk allele in rs918592, may be in LD with one or more of the alleles identified in “Block 1” and “Block 2” in FIG. 4. Thus, the secondary alleles may play a role in providing for the detection of the genetic risk factor for stroke susceptibility in a subject. It is further contemplated that any other SNPs, within the PDE4D gene or otherwise, may be in LD with any of the exemplary risk alleles (i.e., rs918592, rs153031, rs152312, rs12188950, rs27224, rs153067, rs42222, and rs918590) and exhibit an increased or decreased stroke association.

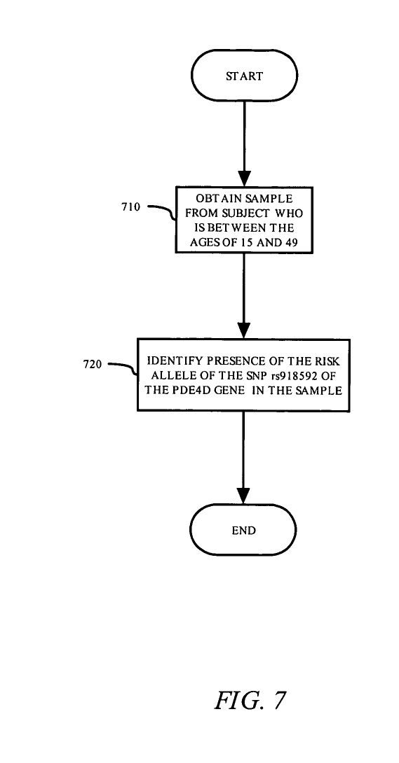

In a preferred embodiment, shown in FIG. 7, a method for identifying a subject at risk for early onset stroke is provided. The method includes a first step 710 of obtaining a sample from a subject, the subject being between the ages of 15-49. A second step 720 of the method includes the step of detecting the presence of a risk allele of the SNP rs918592, within the sample. As stated previously, this risk allele of the present invention provides a unique specificity for the identification of stroke susceptibility within an age subpopulation. This level of specificity has not been observed within previously identified risk alleles of the PDE4D gene, thus giving the risk allele of the current invention a significant advantage over previously known genetic markers indicating a susceptibility to stroke. Alternatively, the second step of the current method may include the detection of the presence of a risk allele from the PDE4D gene. The risk allele of the PDE4D gene may exhibit a decreased or increased association as compared to that shown by rs918592.

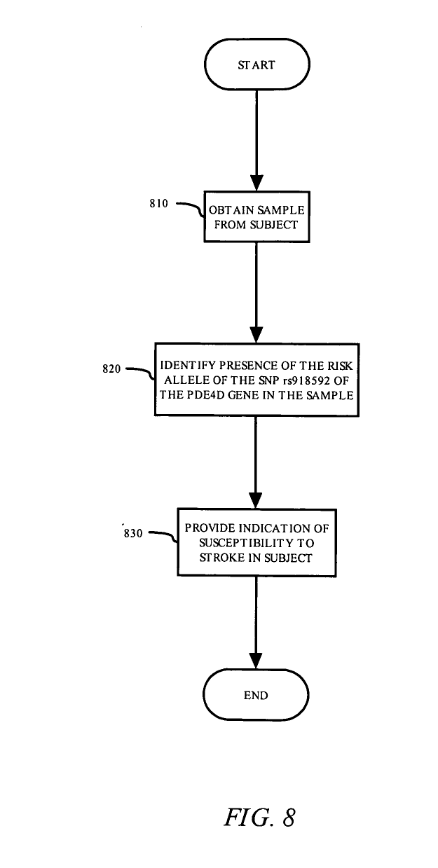

The present invention also provides a method for screening a subject for a susceptibility to stroke. FIG. 8 illustrates the method which includes a first step 810 of obtaining a sample from the subject and a second step 820 of detecting the presence of the risk allele within SNP rs918592 present in the sample. In step 830, from the detection of the risk allele, an indication of susceptibility to stroke is provided. The providing of an indication of susceptibility to stroke may include disclosing to the subject the presence of the risk allele that indicates a susceptibility to stroke. It is contemplated that the screening method may result in the indication that the risk allele is not present in the sample from the subject. Alternatively, the second step of the current method may include the detection of the presence of a risk allele from the PDE4D gene. The risk allele of the PDE4D gene may exhibit a decreased or increased association as compared to that shown by rs918592.

In the current embodiment of the method shown in FIG. 8, the particular type of stroke is an ischemic stroke. The ischemic stroke screened for may include early on-set stroke. It is contemplated that the screening of risk for an ischemic stroke may include various sub-types of ischemic stroke, such as Atherosclerotic, Lacunar, Nonlacunar stroke of underdetermined etiology, and the like, as previously identified. The method may further comprise the step of providing preventative and/or treatment options to the subject. The subject and sample employed in this method may be similar to those employed and described above and for previously described methods.

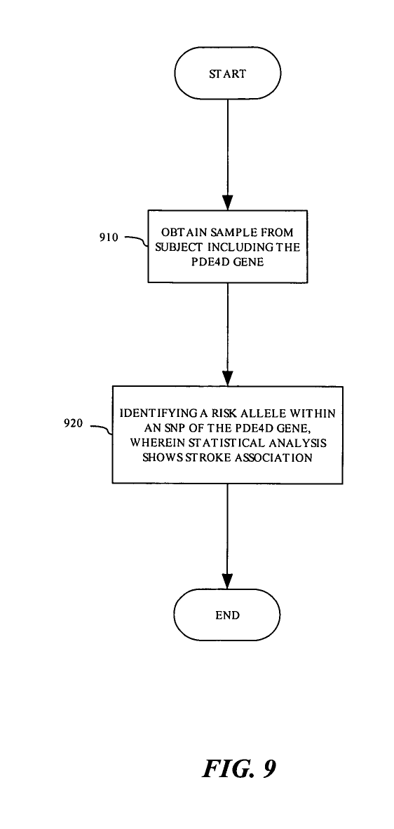

In a preferred embodiment shown in FIG. 9, a method for identifying a stroke associated genetic marker includes a step 910 of obtaining a sample from a human subject wherein the sample includes a PDE4D gene. The subject and sample may be similar to those described previously. After the sample is obtained a next step 920 is identifying a risk allele within an SNP of the PDE4D gene showing association to stroke through statistical analysis (described below) by having a p value ≦0.05 and an OR ≧1. As has been described previously, a genetic marker exhibiting such p and OR values shows a strong association with stroke susceptibility in a subject. It is contemplated that the p and OR values for a genetic marker may vary, such as having a p value which is greater than 0.05 and an OR value that is less than 1, without departing from the scope and spirit of the present invention. In a current embodiment, the method may include the identification of the risk allele “A” within SNP rs918592 within the PDE4D gene or the identification of other risk alleles within the gene.

Research Design and Methods

The following provides a detailed description of the exemplary research design techniques and methodologies employed for the current invention. It is to be understood that various design techniques, methodologies, other tools, and the like, as contemplated by those of ordinary skill in the art may be employed without departing from the scope and spirit of the present invention.

Study Subjects

The Stroke Prevention in Young Women Study 2 (SPYW2) is a population-based case-control study that was initiated to examine genetic risk factors for ischemic stroke in young women. The term “population-based” indicates that the cases and their comparison group were identified from the same defined population, which included all of Maryland (except the far Western panhandle), Washington D.C., and the southern portions of both Pennsylvania and Delaware. Cases were 239 female patients 15-49 years of age with a first cerebral infarction as identified by discharge surveillance at 51 regional hospitals and through direct referral by regional neurologists. The methods employed for discharge surveillance, chart abstraction, and case adjudication have been described previously. (Johnson C J, Kittner S J, McCarter R J, et al. Interrater reliability of an etiologic classification of ischemic stroke. Stroke. 1995; 26:46-51; Kittner S J, Stern B J, Feeser B R, et al. Pregnancy and the risk of stroke. N Engl J Med. 1996; 335:768-774; Kittner S J, Stern B J, Wozniak M, et al. Cerebral infarction in young adults: the Baltimore-Washington Cooperative Young Stroke Study. Neurology. 1998; 50:890-4.) The adjudication of stroke cases was performed blinded to genetic information. Stroke cases were classified as having a probable, possible or undetermined etiology as described previously. (Johnson C J, Kittner S J, McCarter R J, et al. Interrater reliability of an etiologic classification of ischemic stroke. Stroke. 1995; 26:46-51; Kittner S J, Stern B J, Feeser B R, et al. Pregnancy and the risk of stroke. N Engl J Med. 1996; 335:768-774.) Using predetermined exclusions modified from the Siblings With Ischemic Stroke Study (SWISS) protocol, (Meschia J F, Brown R D Jr, Brott T G, et al. The Siblings With Ischemic Stroke Study (SWISS) protocol. BMC Med Genet. 2002; 3(1):1. Epub Feb. 12, 2002) cases with the following characteristics were excluded: sickle cell disease (1), CNS vasculitis by angiogram and clinical criteria (3), post-radiation arteriopathy (1), endocarditis (3), neurosyphillis (1), mechanical prosthetic heart valves (2), left atrial myxoma (1), and cocaine use in the 48 hours prior to their stroke (3). Control subjects were 212 women without a history of stroke, identified by random digit dialing and were frequency matched to the cases by age and geographic region of residence. One control was excluded from analyses based on a history of sickle cell disease. Thus, the sample for genetic analyses consisted of 224 cases and 211 controls.

Cases and controls were grouped into the following race/ethnic categories: Caucasian (non-Hispanic) (n=95 cases and 99 controls), African-American (n=105 cases and 91 controls), and other (including Hispanic, Asian, American-Indian, etc.) (n=24 cases and 21 controls). Because of the small size and heterogeneity of the latter group, it was not analyzed separately, but was included within the combined total study group (n=224 cases and 211 controls). Haplotype analyses were conducted only on the Caucasian (non-Hispanic) and the African-American groups.

Previously Identified SNPs

Stroke risk was elevated using the following SNPs: rs12188950 (deCODE SNP 45), rs152312 (SNP 41), rs153031 (SNP 42)), rs966221 (SNP 83), and rs1396476 (SNP 89). Each of these SNPs are in the region near the alternative forms of exon 1. SNPs were also chosen from the NCBI database based upon their location in the PDE4D gene and their allele frequencies (minor allele frequency >0.05). Thus, rs27224, rs153067, rs42222, rs918590, rs918592, rs294496, rs525099, rs929820, and rs1498606, were included in this study.

Novel SNP Discovery

Screening for novel polymorphisms was performed on a subset of stroke cases and controls including both African-Americans and Caucasians (24 unrelated individuals for each group, in total 96 individuals). Because the PDE4D gene locus is very large and coding SNPs tend to occur very infrequently in exons, (Gretarsdottir S, Thorleifsson G, Reynisdottir S T, et al. The gene encoding phosphodiesterase 4D confers risk of ischemic stroke. Nat Genet 35: 131-138, 2003) computational VISTA analysis was performed. Aiming to identify potentially functional non-coding variants, highly evolutionarily conserved regions were selected throughout the entire human PDE4D gene that were revealed in the VISTA map as the targets, with particular emphasis on the PDE4D7 isoforms and PDE4D1/2 isoforms, the 5′ flanking regions (5-kb) for all PDE4D isoform-specific promoters, and the exon-intron junctions.

VISTA Alignments

Postulating that highly evolutionary conserved genomic regions among species are likely to be functionally important, the VISTA Web server (Mayor C, Brudno M, Schwartz J R, et al. VISTA: visualizing global DNA sequence alignments of arbitrary length. Bioinformatics 16:1046-1047, 2000.) was used (http://www-gsd.lbl.gov/VISTA/) to align genomic sequences in the in silico analysis of global sequence conservation on the PDE4D gene locus. The following polynucleotide sequences were aligned: human (UCSC human genome assembly 2004 May chr5: 59968082..58282468), mouse (UCSC mouse genome assembly 2004 May chr13: 104923788..106765297), and rat (UCSC rat genome assembly 2003 June chr2: 39230274..41294264) genomic sequences containing the entire PDE4D gene and its upstream (100 kb) and downstream (20kb) intergenic sequences. Transposable elements in the mammalian sequences were masked. Human sequence and its annotation were used as the base sequence. Pairwise sequence comparisons were calculated with a threshold of 75% identity in a 100-bp window. All segments with a conservation >=80% over a 100 bp window or a conservation >=75% over 100 bp but within proximal 5 kb of the 5′-flanking regions for each PDE4D isoform-specific promoter, were selected as the targets for the DHPLC analysis.

DHPLC Analysis

PCR primers were designed to amplify the targeted regions. Genomic DNA (10 ng) was amplified by PCR using AmpliTaq Gold (Applied Biosystems, Foster City, Calif.), denatured at 94° C. for 5 min, and slowly re-annealed at room temperature. Samples were then analyzed in the WAVE DNA Fragment Analysis System (Transgenomic Inc, Omaha, Nebr.) based on denaturing high-performance liquid chromatography (DHPLC), using a buffer gradient and oven temperature controlled by the WAVEMaker software. Heterozygous amplicons were identified by elution profiles. For each type of elution profile, 3 representative heterozygous samples were further analyzed on an ABI 3100 automatic sequencer (Applied Biosystems) in the forward and reverse directions. Sequence trace files were analyzed using the Mutation Surveyor software (SoftGenetics, State College, PA) to locate nucleotide variants and were verified by manual inspections. Polymorphic PCR amplicons (with minimal of 3 individuals for a DHPLC heteroduplex elution profile) were subjected to further sequence analysis.

Genotyping Methods for the Case/Control Population

Of the SNPs meeting frequency criteria (minor allele frequency >0.05), the following genotyping selection priority criteria were applied: 1) greater minor allele frequencies, 2) distribution throughout the gene, 3) VISTA peaks, and 4) deCODE positive SNPs. Twenty-four SNPs were genotyped in our stroke cohort (Table 2).

Genotyping was conducted using DNA isolated from whole blood using the QIAamp DNA Blood Maxi Kit (Qiagen, Valencia, Calif.). SNP typing was performed by one of the following methods.

1. The first method types 12 SNPs simultaneously and was developed for a SNPstream Ultra-High Throughput machine (Beckman Coulter, Inc., Fullerton, Calif.) Sequences surrounding the SNPs were obtained from GenBank (http://www.ncbi.nlm.nih.gov/Genbank/index.html) and submitted to Autoprimer.com (Beckman Coulter, Inc.). For each SNP, 3 primers were designed, two for PCR amplification and an internal primer with a 5′ DNA sequence tag. Twelve pairs of primers were used to initiate PCR amplification. The free primers were removed by enzymatic digestion using Exonuclease I and Shrimp Alkaline Phosphotase (Beckman Coulter, Inc.). The internal primers were used to initiate a sequencing reaction that adds one labeled base for the alternative nucleotides of each SNP to have distinct labels. The labeled products are separated on a SNP-IT plate consisting of 384 mini-arrays with 16 spots each (Beckman Coulter, Inc.). For each individual DNA sample, 16 spots hybridized to the two homozygotes, the heterozygote, a negative control and the 12 labeled primers associated with the 12 SNPs. Thus, every PCR and labeling reaction has internal controls to confirm the success of the reactions and the appropriateness of the fluorescent outputs for each DNA sample.

2. The second method was the Taqman method (Applied Biosystems). This method is based on four primers, two flanking the SNP that are used to amplify the DNA surrounding the SNP and two, one for each alternative allele, that were labeled with different fluorescent dyes. The original form of the labeled primer has a quencher in close proximity to the dye. However, when the exonuclease activity of DNA polymerase disrupts the primer hybridized to the single strand DNA during the PCR, then quencher and the dye are released and the fluorescence can be measured. The reaction conditions were per manufacturer's instructions included with each individual primer set.

Analyses

All statistical analyses were performed using SAS®, Version 9.1 (SAS Institute, Cary, N.C.). Means were compared by t tests and proportions by ×2 tests. Probability values are based on two-sided tests. For the primary analysis, an additive model was considered to test the effect of genotype of stroke. Odds ratios were computed to depict the increased odds of stroke associated with each additional risk allele. Analyses were conducted in the total population, and in African-American and Caucasian women separately. Dominant and recessive genetic models were also considered as a secondary analysis.

Adjusted odds ratios derived by logistic regression were used to determine whether the presence of the risk allele was associated with an increased risk for stroke after controlling for potential confounders. The minimally adjusted model included age and race. The environmental model included age, race, current cigarette smoking, and use of oral contraceptive pills (OCP). The environmental/vascular risk factor model included age, race, OCP use, hypertension, diabetes mellitus, current cigarette smoking, and history of angina or myocardial infarction (angina/MI). Age, race, current cigarette smoking status, and use of OCP were determined by subject reports (or proxy report, if a participant was unable to answer). Hypertension, and diabetes mellitus were determined by asking study participants (or a proxy) if a physician had ever told them they had the condition.

Significant SNPs underwent additional race-specific stratified analyses to evaluate groups stratified by standard risk factors (age, OCP use, current cigarette smoking, hypertension, diabetes mellitus, and history of angina or myocardial infarction) and stroke subtype (atherosclerotic, cardiac, dissection, lacunar, hematologic, and all other stroke). Tests for interaction were performed using logistic regression.

Because multiple SNPs were considered, a “significant” association may have been observed with any one SNP by chance. However, because many of the SNPs are correlated with each other, a Bonferroni-type adjustment would be too conservative (i.e., the 24 SNPs do not represent 24 independent tests). To account for the non-independence of SNPs, a permutation test was performed, the genotype vector was permuted (comprising of all SNPs with a mean allele frequency greater than 0.05 in both races and among both cases and controls; 16 SNPs satisfied these criteria) among subjects. This has the effect of maintaining the original data structure, but disassociating any phenotype-genotype correlation that may have existed. A distribution of expected p-values was constructed under the null hypothesis by performing an association analysis of the SNPs within each permutation, identifying the smallest p-value from each set of SNP analyses (n=16), and then repeating the permutation and analysis. The data structure was permutated 5,000 times, each time identifying the smallest p-value of the analyzed SNPs (n=16). The multiple comparison-corrected p-value was defined as the proportion of the 5,000 p-values obtained from the permutation that were lower than the p-value observed from the analysis of the real data.

In the exemplary embodiments, it is understood that the specific order or hierarchy of steps in the methods disclosed are exemplary approaches. Based upon design preferences, it is understood that the specific order or hierarchy of steps in the method can be rearranged while remaining within the scope and spirit of the present invention. The accompanying method claims present elements of the various steps in a sample order, and are not necessarily meant to be limited to the specific order or hierarchy presented.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

### REFERENCES

1. Gretarsdottir S, Thorleifsson G, Reynisdottir S T, et al. The gene encoding phosphodiesterase 4D confers risk of ischemic stroke. Nat Genet 35: 131-138, 2003.
2. Dominiczak A F, McBride M W. Genetics of common polygenic stroke. Nat Genet 35: 116-117, 2003.
3. Nilsson-Ardnor S, Wiklund P G, Lindgren P, Nilsson A K, Janunger T, Escher S A, Hallbeck B, Stegmayr B, Asplund K, Holmberg D. Linkage of ischemic stroke to the PDE4D region on 5q in a Swedish population. Stroke. 2005 August; 36(8):1666-71.
4. Meschia J F, Brott T G, Brown R D Jr, Crook R, Worrall B B, Kissela B, Brown W M, Rich S S, Case L D, Evans E W, Hague S, Singleton A, Hardy J. Phosphodiesterase 4D and 5-lipoxygenase activating protein in ischemic stroke. Ann Neurol. 2005 September; 58(3):351-61.
5. Lohmussaar E, Gschwendtner A, Mueller J C, et al. ALOX5AP gene and the PDE4D gene in a central European population of stroke patients. Stroke. 2005 April; 36(4):731-6. Epub Feb. 24, 2005.
6. Bevan S, Porteous L, Sitzer M, et al. Phosphodiesterase 4D Gene, Ischemic Stroke, and Asymptomatic Carotid Atherosclerosis. Stroke. 2005 May; 36(5):949-53. Epub Mar. 31 2005.
7. van Rijn M J, Slooter A J, Schut A F, Isaacs A, Aulchenko Y S, Snijders P J, Kappelle L J, van Swieten J C, Oostra B A, van Duijn C M. Familial aggregation, the PDE4D gene, and ischemic stroke in a genetically isolated population. Neurology. 2005 September 14; [Epub ahead of print].
8. Saleheen D, Bukhari S, Haider S R, Nazir A, Khanum S, Shafqat S, Anis M K, Frossard P. Association of Phosphodiesterase 4D Gene With Ischemic Stroke in a Pakistani Population. Stroke. 2005 October; 36(10):2275-7.
9. Johnson C J, Kittner S J, McCarter R J, et al. Interrater reliability of an etiologic classification of ischemic stroke. Stroke. 1995; 26:46-5 1.
10. Kittner S J, Stem B J, Feeser B R, et al. Pregnancy and the risk of stroke. N Engl J Med. 1996; 335:768-774.
11. Kittner S J, Stem B J, Wozniak M, et al. Cerebral infarction in young adults: the Baltimore-Washington Cooperative Young Stroke Study. Neurology. 1998; 50:890-4.
12. Meschia J F, Brown R D Jr, Brott T G, et al. The Siblings With Ischemic Stroke Study (SWISS) protocol. BMC Med Genet. 2002; 3(1):1. Epub Feb. 12, 2002.
13. Mayor C, Brudno M, Schwartz J R, et al. VISTA: visualizing global DNA sequence alignments of arbitrary length. Bioinformatics 16:1046-1047, 2000.
14. Celermajer D S, Sorensen K, Georgakopoulis D, et al. Cigarette smoking is associated with dose-related and potentially reversible impairment of endothelium-dependent dilatation in healthy young adults. Circulation. 1993; 88: 2149-2155.
15. Zhang S, Day I N, Ye S. Microarray analysis of nicotine-induced changes in gene expression in endothelial cells. Physiol Genomics. April 27, 2001; 5(4): 187-92.
16. Ratan R R. cAMP response element binding protein family transcription factors: the Holy Grail of neurological therapeutics? Ann Neurol. 2004 November; 56(5):607-8.
17. Singh S P, Barrett E G, Kalra R, et al. Prenatal cigarette smoke decreases lung cAMP and increases airway hyperresponsiveness. Am J Respir Crit Care Med. Aug. 1, 2003 1 ;168(3):342-7.
18. Cohen J C, Kiss R S, Pertsemlidis A, et al. Multiple rare alleles contribute to low plasma levels of HDL cholesterol. Science 305: 869-872, 2004.

What is claimed is:

1. A method for identifying susceptibility to stroke in a subject, comprising:
obtaining a sample from a human subject; and
determining if the sample contains a risk allele of SNP rs918592.

2. The method of claim 1, further comprising the step of determining if a secondary allele associated with stroke is in linkage disequilibrium with rs918592.

3. The method of claim 2, wherein the secondary allele is selected from the group consisting of rs153031, rs152312, rs12188950, rs27224, rs153067, rs42222, rs918590, any other SNP in LD with these SNPs and any other SNP in LD with the risk allele.

4. The method of claim 1, wherein the subject is an African-American human female.

5. The method of claim 1, wherein the subject is a Caucasian human female.

6. The method of claim 1, wherein the subject is between the ages of 15-49.

7. The method of claim 1, wherein the subject is a human female selected from the group consisting of Hispanic, Asian, and American-Indian.

8. The method of claim 1, wherein the subject is a smoker.

9. The method of claim 1, further comprising the step of determining if rs9l8592 is associated with ischemic stroke.

10. A method of screening a subject for susceptibility to stroke, comprising:
obtaining a sample from a human subject;
detecting a risk allele of the SNP rs918592; and
providing an indication of susceptibility to stroke based on the detection of the risk allele.

11. The method of claim 10, further comprising the step of determining if a secondary allele associated with stroke is in linkage disequilibrium with the risk allele.

12. The method of claim 11, wherein the secondary allele is selected from the group consisting of rs153031, rs152312, rs12188950, rs27224, rs153067, rs42222, rs918590, any other SNP in LD with these SNPs and any other SNP in LD with the risk allele

13. The method of claim 10, wherein the stroke type is ischemic stroke.

14. The method of claim 10, wherein the subject is an African-American human female.

15. The method of claim 10, wherein the subject is a Caucasian human female.

16. The method of claim 10, wherein the subject is between the ages of 15-49.

17. The method of claim 10, wherein the subject is a smoker.

18. A method for identifying a stroke associated genetic marker, comprising:
obtaining a sample from a human subject, wherein the sample includes a PDE4D gene; and
identifying a risk allele within an SNP of the PDE4D gene showing association to stroke through statistical analysis wherein the risk allele exhibits a p value less than or equal to 0.05 and an OR of 1 or more.

19. The method of identifying a stroke associated genetic marker of claim 18, wherein the risk allele is allele “A” of the SNP rs918592 of the PDE4D gene.

20. The method of identifying a stroke associated genetic marker of claim 18, wherein a secondary allele associated with stroke is in linkage disequilibrium with the risk allele, wherein the secondary allele is selected from the group consisting of rs153031, rs918592, rs152312, rs12188950, rs27224, rs153067, rs42222, rs918590 and any other SNP in LD with these SNPs.

21. The genetic marker of claim 18, wherein the risk allele is associated with ischemic stroke.

22. The genetic marker of claim 18, wherein the human subject is an African-American female.

23. The genetic marker of claim 18, wherein the human subject is a Caucasian human female.

24. The genetic marker of claim 18, wherein the human subject is between the ages of 15-49.

25. The genetic marker of claim 18, wherein the p value may be greater than 0.05 and the OR may be less than 1.
